(19) United States
(12) Patent Application Publication
(10) Pub. No.: US 2007/0191326 A1
(43) Pub. Date: Aug. 16, 2007

(54) Methods for modulating glutamate receptors for treating neuropsychiatric disorders comprising the use of modulators of serum and glucocorticoid inducible kinases

(76) Inventor: Florian Lang, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/592,106

(22) PCT Filed: Feb. 8, 2005
(86) PCT No.: PCT/EP05/01245

§ 371 Date: Sep. 8, 2006

(30) Foreign Application Priority Data
Mar. 11, 2004 (EP) 04005761.4

(51) Int. Cl. A61K 31/573

(52) U.S. Cl. 514/179

(57) ABSTRACT

Modulation of the activity of serum and glucocorticoid inducible kinases to restore glutamate receptor activity. Also disclosed are methods and compounds useful for the detection and treatment of neuropsychiatric disorders.

### FIELD OF THE INVENTION

A method for altering glutamate receptor activity comprising, contacting cells expressing serum and glucocorticoid inducible kinases SGK1, SGK2 or SGK3 with a substance that modulates glucocorticoid inducible kinases. Furthermore the invention relates to the diagnosis and treatment of diseases related to glutamate receptor up- or down-regulation.

### BACKGROUND OF THE INVENTION

Neurons continually modify the relative expression, function, and subcellular localization of neurotransmitter receptors to maintain and fine-tune neurotransmission. Among the excitatory receptor systems modified are members of the AMPA family of the ionotropic glutamate receptor (GluR) that include subunits GluR1 thru GluR4. AMPA receptors are involved in a variety of diseases as epilepsy, Alzheimer's disease, Parkinson's disease, and Rasmussen's encephalitis. Rasmussen's encephalitis is a progressive disorder that is characterized by severe epilepsy, hemiplegia, dementia and inflammation of the brain. There is evidence that Rasmussen's encephalitis develops if the patient raises antibodies against GluR3. It has been established that GluR3 antibodies are able to activate AMPA receptors in cortical neurons and that the region of GluR3 with which the self-antibodies interact lies within the agonist binding site (Twyman et al., 1995). Neuronal excitation induced by receptor activation may therefore precipitate the epileptic seizures which characterize the disease. However, many issues remain to be solved in order to understand the mechanism driving neuropsychiatric diseases.

Glutamate receptors are the most important mediators of excitatory signal transduction in the central nervous system (M Sheng, T. Nakagawa, Nature 417, 601, 2002). They activate a multitude of biochemical pathways in postsynaptic neurons eventually leading to postsynaptic neuronal plasticity. Changes in synaptic strength can occur by changing the activity and/or abundance of postsynaptic AMPA receptors. Hippocampal phosphatidylinositol-3-Kinase (Pl3-K) is activated during long-term potentiation and complexed with synaptic AMPA receptors (P. P. Sanna, et al., J. Neurosci, 22, 3359, 2002; M. Passafaro, V. Piech and M. Sheng, Nat. Neurosci, 4, 917, 2001; H. Y. Man, et al., Neuron 39, 611, 2003). However, the signaling pathway from Pl3-K to AMPA receptor abundance in the cell membrane remained elusive. Among the downstream signaling molecules of Pl3-K is the 3-phosphoinositide-dependent kinase (PDK) which phosphorylates and thus activates protein kinase B and all three members of the serum and glucocorticoid-inducible kinase family, SGK1, SGK2 and SGK3 (F. Lang, P. Cohen, Sci. STKE. 108, RE17, 2001). SGK1, SGK2 and SGK3 have all three been shown to regulate the renal epithelial Na⁺ channel ENaC by increasing the abundance of the channel protein in the plasma membrane (F. Lang et al., Cell. Physiol. Biochem. 13, 41, 2003; D. Pearce, Cell. Physiol. Biochem. 13, 13-20, 2003; F. Verrey, J. Loffing, M. Zecevic, D. Heitzmann, O. Staub, Cell. Physiol. Biochem. 13, 21, 2003). As all three kinases are abundantly expressed in the brain (T. Kobayashi, P. Cohen, Biochem J. 339, 319, 1999; S. Waldegger, P. Barth, J. N. Jr. Forrest, R. Greger, F. Lang, Proc. Natl. Acad. Sci. U.S.A 94, 440, 1997), we hypothesized that they may participate in the regulation of AMPA receptors.

SGK1 has been shown to be regulated through Insulin like growth factor IGF1, insulin and through oxidative stress via a signal cascade involving phosphoinositol-3-kinase (Pl3 kinase) and phosphoinositol-dependent kinase PDK1 (Kobayashi & Cohen 1999, Park et al. 1999, Kobayashi et al. 1999). The activation of SGK1 through PDK1 involves phosphorylation of Serine 422. It has furthermore been shown, that a mutation of ser 422 to aspartate (^{S422D}SGK1) results in a continuatively activated kinase (Kobayashi et al. 1999).

For the measurement of glucocorticoid inducible kinase SGK1 activity various assay systems are available. In scintillation proximity assay (Sorg et al., J. of. Biomolecular Screening, 2002, 7, 11-19) and flashplate assay the radioactive phosphorylation of a protein or peptide as substrate with γATP will be measured. In the presence of an inhibitory compound no or decreased radioactive signal is detectable. Furthermore homogeneous time-resolved fluorescence resonance energy transfer (HTR-FRET), and fluorescence polarization (FP) technologies are useful for assay methods (Sills et al., J. of Biomolecular Screening, 2002, 191-214). Other non-radioactive ELISA based assay methods use specific phospho-antibodies (AB). The phospho-AB binds only the phosphorylated substrate. This binding is detectable with a second peroxidase conjugated anti sheep antibody by chemiluminescence (Ross et al., 2002, Biochem. J., immediate publication, manuscript BJ20020786).

Earlier results showed that SGK1 is a potent stimulator of the renal epithelial Na⁺-channel (De la Rosa et al. 1999, Boehmer et al. 2000, Chen et al. 1999, Naray-Fejes-Toth et al. 1999, Lang et al. 2000, Shigaev et al. 2000, Wagner et al. 2001).

Another finding related to SGK1 was that a single nucleotide polymorphism (SNP) in exon 8 with nucleotide combinations of (CC/CT) and an additional polymorphism in intron 6 (CC) are associated with increased blood pressure (Busjahn et al. 2002) and from this it was concluded that SGK1 may be important to blood pressure regulation and hypertension.

Because increased activity of SGK1 correlates with renal epithelial Na⁺ channel activity which leads to hypertension through the increase of renal resorption of sodium (Lifton 1996; Staessen et al., 2003; Warnock 2001), it was conclusive that depending on the combination of allelic variants of SGK1 an increase in renal Na⁺-resorption may occur which in turn will increase the blood pressure (Busjahn et al. 2002).

### SUMMARY OF THE INVENTION

The current application unexpectedly demonstrates that several isoforms of the serum and glucocorticoid inducible kinases are powerful regulators of glutamate receptors.

Little is known about the regulation of glutamate receptors and this invention delivers the unexpected result that all three members of the serum and glucocorticoid-inducible kinase family, SGK1, SGK2 and SGK3 are involved in glutamate receptor regulation. Because glutamate receptors are the most important mediators of excitatory signal transduction in the nervous system, their up- or down-regulation has been discussed in a considerable number of neuro-psychiatric diseases. Thus the invention delivers a method for determining the progression, regression or onset of a neuropsychiatric disease by measuring the up-regulated expression of SGK1, SGK2 or SGK3 in tissue samples and specimens in conjunction with the status of the glutamate receptors activity.

SGK has been shown for the first time to participate in the Pl3-K-dependent regulation of AMPA receptors being known to confer trafficking, synaptic plasticity and memory consolidation.

While SGK3 is a powerful regulator of GluR1, SKG1 is involved in GluR6 activation. SGK3 enhances the abundance of GluR1 in the plasma membrane and increases GluR1-mediated glutamate-induced currents.

GluR6 does not interact with SGK1 via the SGK1 recognition site RXRXXS/T thus a new mechanism affecting unknown amino acid sequences may be involved.

Another finding of this invention that is that GluR6 is an essential subunit of the kaniate receptors and that regulation of GluR6 via SGK1 is involved in the regulation of kainate receptor trafficking, synaptic plasticity and neuronal excitability.

Thus influencing the glutamate receptor subunit GluR6 activity by modulation of SGK1 may be a method to target KARs which are abundantly expressed in brain regions involved in learning and memory, such as the hippocampus, as well as in motoric and motivational aspects of behavior, such as basal ganglia and cerebellum.

It is furthermore shown that to a lesser extent SGK2 but not SGK1 increases glutamate-induced currents by enhancing the abundance of the AMPA subunit GluR1 protein in the cell membrane of Xenopus oocytes expressing rat GluR1.

Modulation of SGK1 is especially useful when applied to a clinically relevant phenotype or genotype which is defined by a single nucleotide polymorphism of the SGK1 gene. Therefore the analysis of a polymorph SGK1 SNP variant in samples derived from an individual in need of treatment may be another application. Furthermore the invention delivers a method to determine the progression, regression or onset of a disease by measuring the expression of SGK1. Samples taken from the diseased individuals may furthermore allow the analysis of selected SGK1 SNP variants and their correlation with predisposition for a disease.

Another aspect is related to screening methods for identifying new drug candidates that modulate diseases related to SGK1, SGK2 or SGK3.

Modulators especially useful are compounds that interfere with SGK1 function thus resulting in down-regulation of glutamate receptor activity. Inhibitors of SGK1 are especially useful to treat subjects suffering from symptoms of diseases selected from the group of: Epilepsy, stroke, posttraumatic behavioral disorders, anxiety, schizophrenia, bipolar disorders, depression, hepatic enzephalopathy, morbus hämolyticus neonatorum, addiction, alcoholism, HIV-enzephalopathy, neurodegenerative disorders, extrapyramidal motor disturbance, ataxia, amyotroph lateralsklerosis, M. Alzheimer, macula degeneration and deafness.

The drug screening method performed according to this invention has led to the discovery of SGK1, SGK2 or SGK3 directed therapeutic compounds.

Two different classes of compounds, one belonging to the class of Acylhydrazone derivatives and the other belonging to Pyridopyrimidine derivatives have been identified. Selected SGK1 inhibiting compounds in pharmaceutical compositions comprising a pharmaceutically effective carrier, excipient or diluent are useful for the treatment of fore-mentioned diseases. It is central to this invention that the screening methods used to identify new drugs with the desired therapeutic profile are not restricted to the compounds disclosed in this application. Moreover it is evident to the expert that a one step approach or a two step approach for screening of SGK1, SGK2, SGK3 modulating compounds may be useful to apply. The first step of such a screening includes the identification of compounds that interfere with the SGK kinase activity. Various assay formats are available and a preferred assay uses the measurement of SGK catalyzed radioactive phosphorylation of a protein or peptide as substrate together with the γATP. In the presence of an SGK inhibitory compound no or decreased radioactive signal is detectable. In a second readout system the SGK1 inhibiting compounds are monitored for their potential to restore glutamate receptor activity, however measuring other read-out activities may be useful as well.

### DETAILED DESCRIPTION OF THE INVENTION

Glutamate receptors activate a multitude of biochemical pathways in postsynaptic neurons eventually leading to postsynaptic neuronal plasticity thus an important aspect of the current invention is the teaching how the glutamate receptors are regulated.

The study of regulation of GluR1 required coexpression together with the various SGK isoforms. For testing SGK1, SGK2 or SGK3 have been expressed together with the AMPA receptor subunit GluR1 in Xenopus oocytes. A non-desensitizing GluR1 mutant, GluR1(L479Y) (Y. Stern-Bach, S. Russo, M Neumann, C. Rosenmund, Neuron 21, 907, 1998), has been used for the experiments.

As illustrated in FIG. 1, the protein membrane abundance of GluR1 is significantly increased in Xenopus oocytes expressing GluR1 together with SGK3 as compared to the GluR1 protein abundance in oocytes expressing GluR1 alone. GluR1 protein abundance tended to be higher following coexpression of SGK2, while coexpression of SGK1 was without effect.

The protein abundance was paralleled by similar effects on glutamate-induced currents (FIG. 2). The glutamate-induced currents were significantly larger in Xenopus oocytes expressing GluR1 together with SGK3 than in Xenopus oocytes expressing GluR1 alone. The glutamate-induced currents in Xenopus oocytes coexpressing SGK2 were significantly smaller than those in SGK3-expressing oocytes, but significantly larger than currents in Xenopus oocytes expressing GluR1 alone. Coexpression of SGK1 did not significantly modify GluR1-mediated currents.

The present observations revealed a novel mechanism in the regulation of the GluR1 subunit of AMPA receptors. The delivery of GluR1 to the neuronal surface is regulated by activation of NMDA receptors, leading to Ca²⁺ entry (M. Sheng, M. J. Kim, Science 298, 776, 2002) with subsequent activation of Pl3-kinase (M. S. Perkinton, J. K. Ip, G. L. Wood, A. J. Crossthwaite, R. J. Williams, J. Neurochem. 80, 239, 2002). Activation of Pl3-kinase triggers a signaling cascade eventually leading to activation of SGK3, which then enhances the protein abundance of GluR1 in the cell membrane. SGK3 leads to a stabilized GluR1 in the membrane thus preventing its retrieval and subsequent degradation and/or enhances trafficking of protein to the cell membrane. Therefore the present observations describes for the first time that SGK2 and SGK3 substantially contributes to the fine tuning of GluR1 abundance.

According to the present findings SGK3 is expected to participate in GluR1-dependent neuronal function. GluR1 is dominant over GluR2 in heterodimeric GluR1-GluR2 receptors (Y. Hayashi, et al., Science 287, 2262, 2000; S. Shi, Y. Hayashi, J. A. Esteban, R. Malinow, Cell 105, 331, 2001), is required for hippocampal CA1 long-term potentiation (D. Zamanillo, et al., Science 284, 1805, 1999), and participates in the generation of spatial memory (H. K. Lee, et al., Cell 112, 631, 2003; D. Reisel, et al., Nat. Neurosci. 5, 868, 2002).

To test for regulation of GluR3 by the SGK isoforms the AMPA receptor subunit GluR3 was expressed the in Xenopus oocytes with or without coexpression of either SGK1, SGK2 or SGK3. Glutamate-induced currents were significantly smaller in Xenopus oocytes (FIG. 5) expressing GluR3 together with SGK2 than in Xenopus oocytes expressing GluR3 alone while coexpression with the related protein kinase B (PKB) was without significant effect. SGK1 and SGK3 similarly reduced the current amplitude but were less effective than SGK2.

Administration of dexamethasone, a known modulator of SGK activity, for 8 or 20 days led to a significant increase of GluR6 protein abundance in the mouse hippocampus CA3 neurons (FIG. 6) as seen in brain slices stained with GluR6 polyclonal antibody. Staining hippocampus CA3 neurons with MAP2 antibody, which is a marker for synaptic sites, identified enhanced GluR6 staining at synapses.

Therefore, it was concluded that GluR6 abundance is enhanced by dexamethasone at synaptic sites in hippocampal CA3 neurons. However, it cannot be distinguished between pre- or postsynaptic expression of GluR6.

GFAP, which specifically stains astrocytes, revealed that GluR6 abundance in dexamathasone treated animals is not enhanced at astrocytes compared to control animals (FIG. 6). This result is consistent with predictions based on in situ hybridisation studies which have shown that SGK1 is not expressed in astrocytes (Waerntges et al.).

To test for a functional link between SGK1 and GluR6, the rat KA receptor subunit GluR6 was expressed in Xenopus oocytes with or without coexpression of either SGK1, SGK2 or SGK3. As illustrated in FIG. 4, the protein abundance of GluR6 is significantly enhanced in Xenopus oocytes expressing GluR6 together with SGK1 as compared to the GluR6 protein abundance in oocytes expressing GluR6 alone. A smaller but still statistically significant effect on GluR6 protein abundance was observed following coexpression of SGK2 or SGK3, while coexpression with the related protein kinase B (PKB) was without significant effect. Similar to protein abundance, glutamate-induced current was significantly larger in Xenopus oocytes expressing GluR6 together with SGK1 than in Xenopus oocytes expressing GluR6 alone as shown in FIG. 3. Again, SGK2 and SGK3 similarly stimulated the current but were significantly less effective than SGK1.

The present observations reveal a novel mechanism in the regulation of the GluR6 subunit of KA receptors. The kainate receptors assembled with the GluR6 subunit are important for the sensitivity of CA3 and CA1 pyramidal neurons to kainate and domoate (Bureau et al. 653-63). GluR6 is unlikely to be the immediate target protein for SGK1 because GluR6 does not contain the SGK1 recognition site RXRXXS/T. However, it can not be excluded that SGK1 recognizes other sites than this known amino acid sequence. The membrane protein stargazin has been shown to be critical for guiding AMPA receptors to the cell surface and for targeting them specifically to postsynaptic sites. Stargazin contains the SGK1 recognition site. However, it has been recently published that KARs are not regulated by stargazin (Chen 2003). Therefore, it is not expected that SGK1 regulates GluR6 via stargazin which we confirmed by coinjection experiments in oocytes (data not shown).

The inventive regulatory mechanism involving the new identified kinases is a powerful regulator of GluR6. SGK1 enhances the abundance of GluR6 in the plasma membrane and increases GluR6 mediated glutamate-induced currents. Thus, SGK1 participates in the regulation of kainate receptor trafficking, synaptic plasticity and neuronal excitability.

### BRIEF DESCRIPTION OF THE DRAWINGS

FIG. 1: Increase of GluR1 subunit protein abundance in the cell membrane of Xenopus oocytes coexpressing SGK.

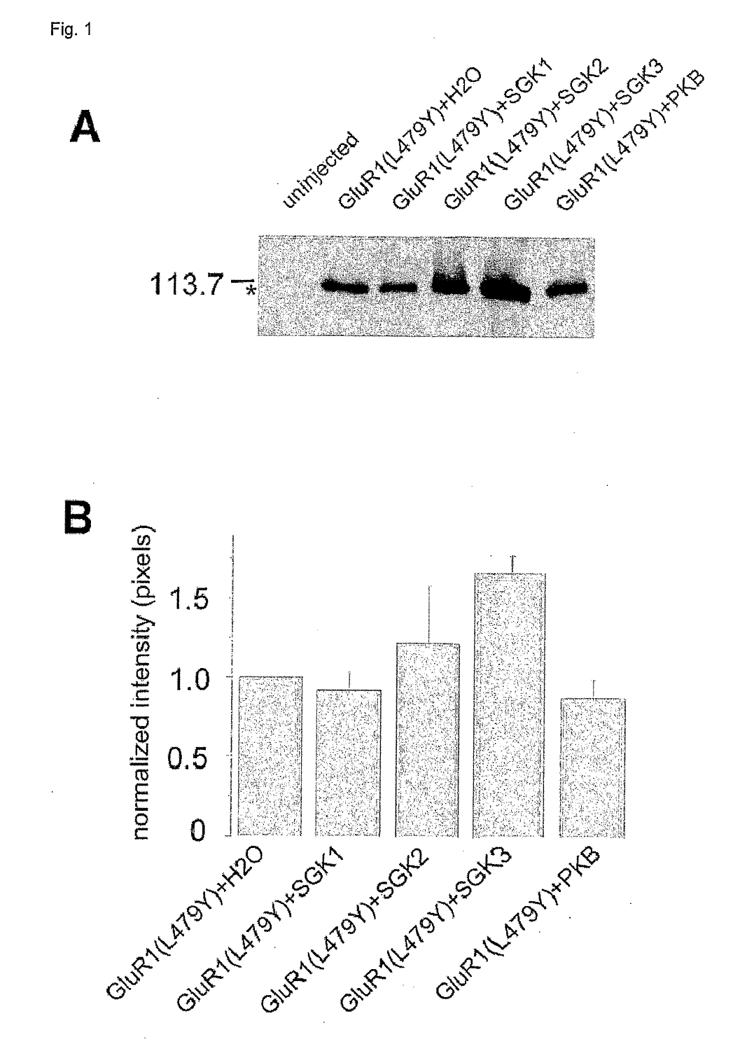

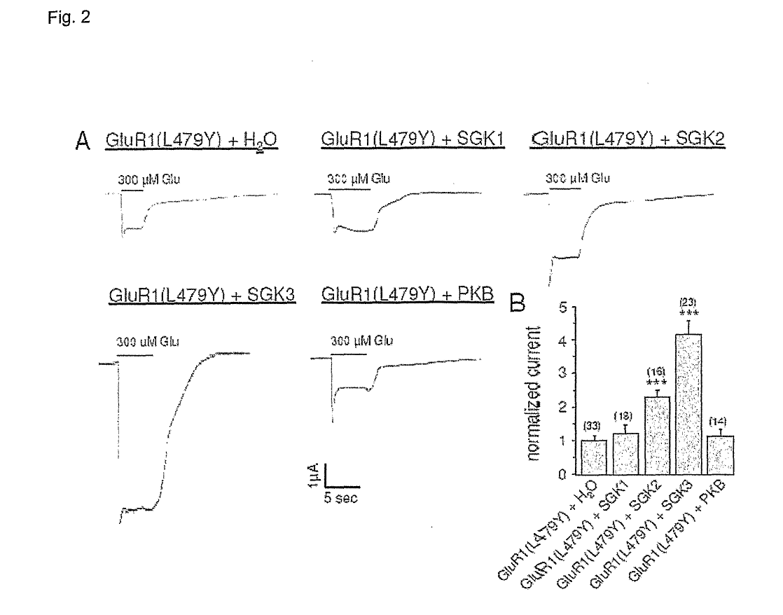

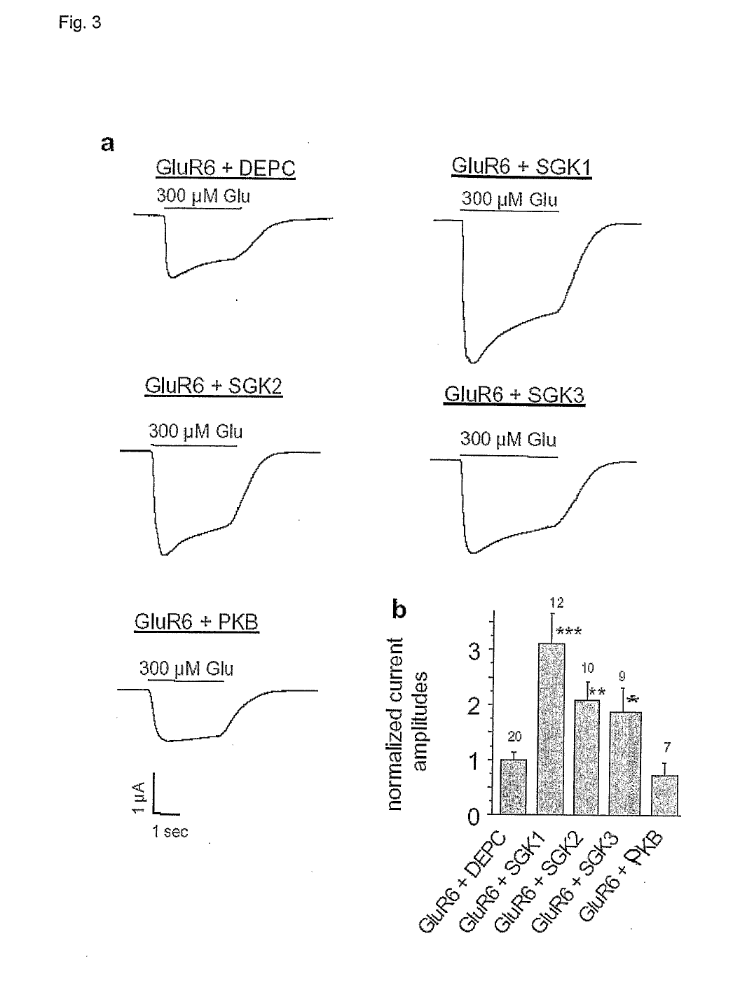

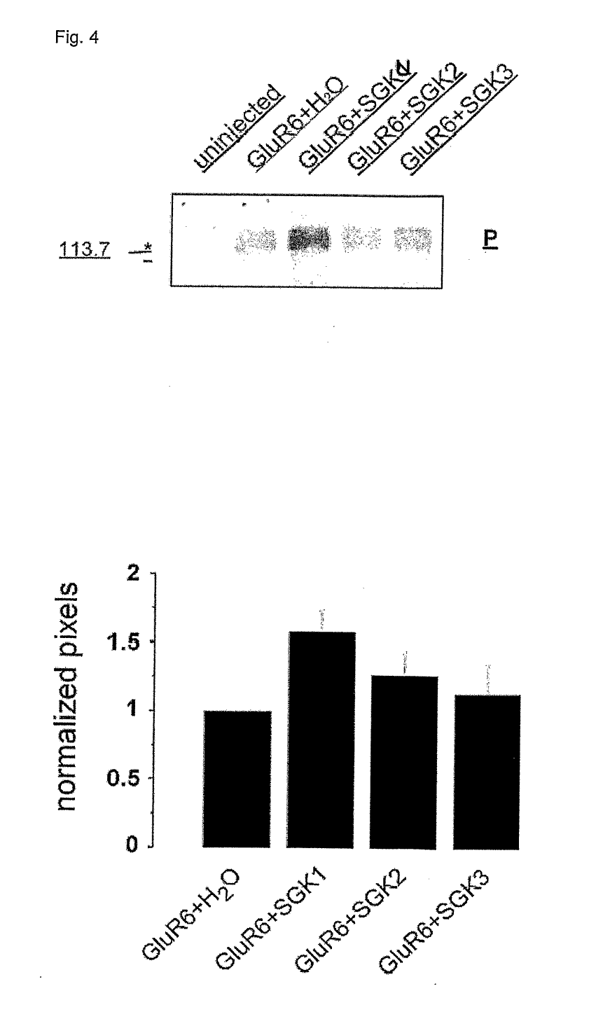

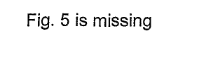

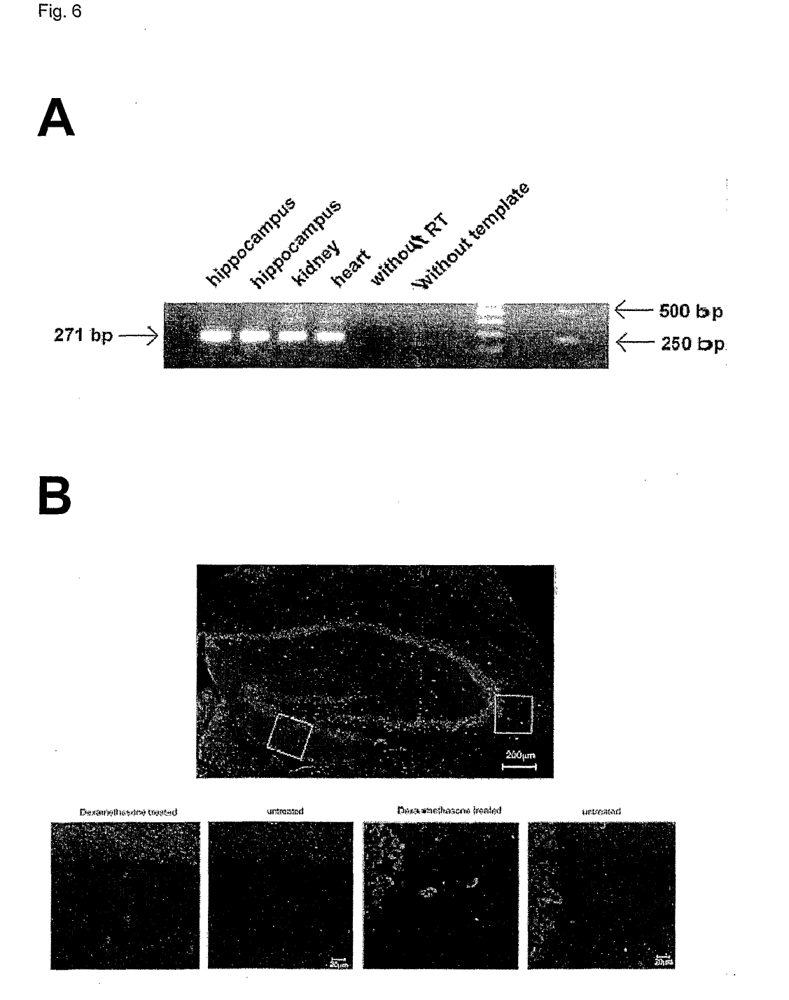

(A) Representative Western blot. For the detection of GluR1, primary rabbit immuno affinity-purified anti-GluR1 antibody (1 μg/μl, Upstate) was used. For the detection of β-tubulin, primary mouse monoclonal anti-β-tubulin antibody (1:250, Santa Cruz) was used. GluR1 protein has an apparent molecular weight of ˜105 kDa. (B) Bar graph showing relative abundance of GluR1 plasma membrane protein. The band intensities were quantified by arithmetic analysis using the software Scion image. The values of three different blots from different batches were used for the statistical analysis. Significant change (p<0.05) is indicated by 1.

FIG. 2: Increase in GluR1 currents by SGK2 and SGK3 isoforms but not by SGK1 and PKB.

Representative (A) current traces measured in Xenopus oocytes in response to superfusion with 300 μM glutamate in ND96 Ringer solution. Oocytes were injected with GluR1 cRNA (4 ng/oocyte) or together with SGK cRNA (6 ng/oocyte) (B) GluR1 current amplitudes in oocytes expressing GluR1(L479Y)+DEPC—H2O, GluR1(L479Y)+SGK1, GluR1(L479Y)+SGK2, GluR1(L479Y)+SGK3 and GluR1(L479Y)+PKB normalized to the GluR1+DEPC—H2O currents. Horizontal scale bars indicate 5 sec and vertical scale bars represent 1 μA. Numbers of oocytes are shown in parenthesis and significant changes (p<0.001) are indicated by ***.

FIG. 3: Increase in GluR6 currents by SGK isoforms but not by PKB.

(a) Representative current traces measured in Xenopus oocytes in response to superfusion with 300 μM glutamate. All currents were measured at 70 mV and after pretreatment of oocytes with ConA to minimize desensitization. (b) GluR6 current amplitudes in oocytes expressing GluR6+DEPC—H₂O (n=20), GluR6+SGK1(n=12), GluR6+SGK2(n=10), GluR6+SGK3(n=9) and GluR6+SGK1(n=7) were measured and are shown normalized to the GluR6+DEPC—H₂O currents. Significant changes upon significance levels of p=0.001 (***), p=0.01 (**) and p=0.05 (*) are indicated.

FIG. 4: Western blot demonstrating SGK-regulated protein expression of the GluR6 subunit.

Plasma membrane protein was labeled with biotinyl-ConA, solubilized, and then streptavidin-precipitated. Samples including controls from uninjected oocytes were separated on a SDS gel, Western-blotted and probed with a immunoaffinity purified antibody directed against a 16 amino acid fragment of an C-terminus of GluR6(Upstate). GluR6 protein has an apparent molecular weight of ˜119 kDa (I).

FIG. 5: Inhibition of GluR3 mediated currents by co-expression SGK2.

(A) Representative current traces measured in Xenopus oocytes in response to superfusion with 300 μM glutamate. All currents were measured at 70 mV and after pretreatment of oocytes with ConA to minimize desensitization. (B) GluR3 current amplitudes in oocytes expressing GluR3+DEPC—H₂O (n=20), GluR3+SGK1 (n=12), GluR3+SGK3(n=10), GluR3+SGK1(n=9), GluR3+SGK3(n=9) and GluR3+SGK1 (n=7) were measured and are shown normalized to the GluR3+DEPC—H₂O currents. Significant changes upon significance levels of p=0.001(***), p=0.01(**) and p=0.05(*) are indicated.

FIG. 6: Expression of GluR6 in hippocampus of Dexamethasone treated and control mice.

(A) Expression of GluR6 in hippocampus, kidney and heart of mice.

Administration of dexamethasone for 8 or 20 days led to a significant increase of GluR6 protein abundance in the mouse hippocampus CA3 neurons (B) as seen in brain slices stained with GluR6 polyclonal antibody. Staining hippocampus CA3 neurons with MAP2 antibody, which is a marker for synaptic sites, identified enhanced GluR6 staining at synapses.

Additional Methods and Materials

### EXAMPLE 1

### Electrophysiological Measurements in Xenopus Oocytes

Oocytes of stages V-VI were surgically removed from the ovaries of Xenopus laevis as described elsewhere (Seebohm, Sanguinetti, Pusch, 2003). Oocytes were injected with either 4 ng of GluR1 or GluR3 or GluR6 cRNA or with or without 6 ng SGK1 or SGK2 or SGK3 or PKB cRNA using a Nanoliter 2000 injector (WPI inc., Florida, USA). Standard two-electrode voltage clamp recordings were performed 5-8 days after cRNA injection with a TurboTec 03 amplifier (npi, Tamm, Germany) and an interface DIGIDATA 1322A from Axon Instruments. Data analyses were done with pClamp 9.0/clampfit 9.0 software (Axon inc.), and Origin 6.0 software (Microcal). Agonist solutions were prepared in ND-96 buffer (in mM, NaCl, 96; CaCl₂, 1,8; KCl, 2,0; MgCl₂, 1,0 and HEPES-NaOH, 5, pH 7.2 with NaOH). Current and voltage electrodes were filled with 3 M KCl and had resistances of 0.5-1.5 MΩ. Oocytes were held at −70 mV and agonist (300 μM glutamate) was applied by superfusion for ˜10 s at a flow rate of 10-14 ml/min. Prior to agonist application, the oocyte was incubated for 8 min in concanavalin A to prevent desensitization.

### EXAMPLE 2

### Labeling of Cell Surface Proteins Using Biotinylated ConA

To identify the fraction of receptor protein inserted in the plasma membrane, surface proteins were tagged with biotinylated ConA and isolated by streptavidin/sepharose-mediated precipitation of the biotinyl-ConA/protein complex. Briefly, intact oocytes were incubated in 10 μM biotinyl-ConA (Sigma, München, Germany) for 30 min at room temperature. After five 10-min washes in normal frog Ringer, intact oocytes were homogenized with a Teflon pestle in H-buffer (20 μl/oocyte; 100 mM NaCl, 20 mM Tris-HCl, pH 7.4, 1% Triton X-100, 1 mM phenylmethylsulphonyl fluoride plus a mixture of proteinase inhibitors (Complete™ tablets, Boehringer)) and were kept at 4° C. for 1 h on a rotator. After centrifugation for 60 s at 16000×g, the supernatants were supplemented with 20 μl washed streptavidin-sepharose beads (Sigma, München, Germany) and incubated at 4° C. for 3 h on the rotator. The streptavidin-sepharose beads were then pelleted by a 120 s spin at 1600×g and washed three times in H-buffer. The final pellets were boiled in 40 μl sodium dodecylsulphate-polyacrylamide gel electrophoresis (SDS-PAGE) loading buffer (0.8 M β-mercaptoethanol, 6% SDS, 20% glycerol, 25 mM Tris-HCl, pH 6.8, 0.1% Bromphenol Blue).measured using an Elisa kit (Mercodia, Uppsala, Sweden).

### EXAMPLE 3

### Gel Electrophoresis and Western Blotting

Proteins from homogenized oocytes were separated by SDS electrophoresis and transferred to nitrocellulose filters. Blots were blocked in 1× PBS containing 5% milk powder for at least 1 hour at room temperature. For the detection of GluR1, GluR3 or GluR6, primary rabbit immunoaffinity purified GluR1, GluR3 or GluR6 antibody (1 μg/μl, Upstate) and secondary horseradish peroxidase-conjugated donkey anti-rabbit antibody (1:1000 dilution, Amersham Bioscience) were used. For verification of protein leves, Ponceau red staining was performed.

### EXAMPLE 4

### SGK1 Modulating Compounds

4.1. Compounds of the General Formula I and Pharmaceutical Useful Derivates, Salts, Solutions and Stereoisomeres Thereof Including Mixtures. 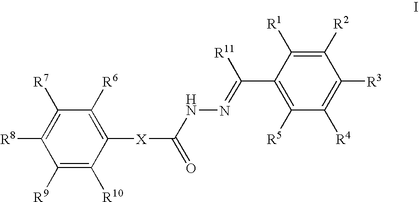
wherein

R¹, R⁵ is either H, OH, OA, OAc or Methyl,

R², R³, R⁴, R⁶, R⁷, R⁸, R⁹, R¹⁰ is either
H, OH, OA, OAc, OCF₃, HaI, NO₂, CF₃, A, CN, OSO₂CH₃, SO₂CH₃, NH₂ or COOH,

R¹¹ H or CH₃,

A Alkyl with 1, 2, 3 or 4 C-atoms,

X CH₂, CH₂CH₂, OCH₂ or —CH(OH)—,

Hal F, Cl, Br or I

Compound according to formula I selected from the following group of compounds:

(3-Hydroxy-phenyl)-acidic acid-(4-hydroxy-2-methoxy-benzyliden)-hydrazid, (3-Hydroxy-phenyl)-acidic acid-[1-(4-hydroxy-2-methoxy-phenyl)-ethyliden]-hydrazid, (3-Methoxy-phenyl)-acidic acid-(4-hydroxy-2-methoxy-benzyliden)-hydrazid.

Phenylacidic acid-(3-fluor-4-hydroxy-benzyliden)-hydrazid,

(4-Hydroxy-phenyl)-acidic acid-(4-hydroxy-2-methoxy-benzyliden)-hydrazid,

(3,4-Dichlor-phenyl)-acidic acid-(4-hydroxy-2-methoxy-benzyliden)-hydrazid, m-Tolyl-acidic acid-(4-hydroxy-2-methoxy-benzyliden)-hydrazid, o-Tolyl-acidic acid-(4-hydroxy-2-methoxy-benzyliden)-hydrazid,

(2-Chlor-phenyl)-acidic acid-(4-hydroxy-2-methoxy-benzyliden)-hydrazid, (3-Chlor-phenyl)-acidic acid-(4-hydroxy-2-methoxy-benzyliden)-hydrazid,

(4-Fluor-phenyl)-acidic acid-(4-hydroxy-2-methoxy-benzyliden)-hydrazid, (2-Chlor-4-fluor-phenyl)-acidic acid-(4-hydroxy-2-methoxy-benzyliden)-hydrazid, (3-Fluor-phenyl)-acidic acid-(4-hydroxy-2-methoxy-benzyliden)-hydrazid, (3-Methoxy-phenyl)-acidic acid-(4-hydroxy-benzyliden)-hydrazid, (3-Methoxy-phenyl)-acidic acid-(4-hydroxy-2,6-dimethyl-benzyliden)-hydrazid, (3-Methoxy-phenyl)-acidic acid-(3-fluor-4-hydroxy-benzyliden)-hydrazid, (3-Methoxy-phenyl)-acidic acid-[1-(4-hydroxy-2-methoxy-phenyl)-ethyliden]-hydrazid, (3-Methylsulfonyloxy-phenyl)-acidic acid-(4-hydroxy-2-methoxy-benzyliden)-hydrazid, (3,5-Dihydroxy-phenyl)-acidic acid-(4-hydroxy-2-methoxy-benzyliden)-hydrazid, (3-Fluor-phenyl)-acidic acid-(3-fluor-4-hydroxy-benzyliden)-hydrazid, (3-Methoxy-phenyl)-acidic acid-(4-acetoxy-2-methoxy-benzyliden)-hydrazid, (3-Trifluormethyl-phenyl)-acidic acid-(4-hydroxy-2-methoxy-benzyliden)-hydrazid, 3-(3-Methoxy-phenyl)-propionsäure-(4-hydroxy-2-methoxy-benzyliden)-hydrazid, (3-Methoxy-phenyl)-acidic acid-(2,4-dihydroxy-benzyliden)-hydrazid, (3-Methoxy-phenoxy)-acidic acid-(4-hydroxy-2-methoxy-benzyliden)-hydrazid, (3-Nitro-phenyl)-acidic acid-(4-hydroxy-2-methoxy-benzyliden)-hydrazid, (3-Methoxy-phenyl)-acidic acid-(5-chlor-2-hydroxy-benzyliden)-hydrazid, (3-Methoxy-phenyl)-acidic acid-(2-hydroxy-5-nitro-benzyliden)-hydrazid, 2-Hydroxy-2-phenyl-acidic acid-(4-hydroxy-2-methoxy-benzyliden)-hydrazid, (3-Methoxy-phenyl)-acidic acid-(2-ethoxy-4-hydroxy-benzyliden)-hydrazid, (3-Brom-phenyl)-acidic acid-(4-hydroxy-2-methoxy-benzyliden)-hydrazid, (3-Methoxy-phenyl)-acidic acid-[1-(4-hydroxy-phenyl)-ethyliden]-hydrazid, (3,5-Difluor-phenyl)-acidic acid-(4-hydroxy-2-methoxy-benzyliden)-hydrazid, (3-Hydroxy-phenyl)-acidic acid-(4-hydroxy-2-methyl-benzyliden)-hydrazid, (3-Hydroxy-phenyl)-acidic acid-(2-ethoxy-4-hydroxy-benzyliden)-hydrazid, (3-Hydroxy-phenyl)-acidic acid-(2-methoxy-4-hydroxy-6-methyl-benzyliden)-hydrazid, (2-Fluor-phenyl)-acidic acid-(2-methoxy-4-hydroxy-benzyliden)-hydrazid

4.2. Compounds of the General Formula II and Pharmaceutical Useful Derivates, Salts, Solutions and Stereoisomeres Thereof Including Mixtures. 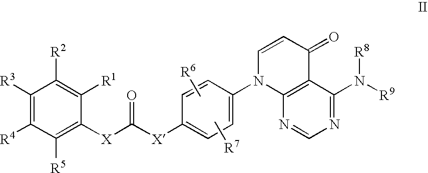
wherein

R¹, R², R³,

R⁴, R⁵ is either H, A, OH, OA, Alkenyl, Alkinyl, NO₂, NH₂, NHA, NA₂, HaI, CN, COOH, COOA,

—OHet, —O-Alkylen-Het, —O-Alkylen-NR⁸R⁹ or CONR⁸R⁹,
two groups selected from R¹, R², R³, R⁴, R⁵ or as well —O—CH₂—CH₂—, —O—CH₂—O— or
—O—CH₂—CH₂—O—,

R⁶, R⁷ is either H, A, Hal, OH, OA or CN,

R⁸, R⁹ is either H or A,

Het

Is a saturated or unsaturated heterocycle with 1 to 4 N-, O- and/or S-atoms, substituted by one or several Hal, A, OA, COOA, CN or Carbonyloxigen (═O)

A Alkyl with 1 to 10 C-atoms, wherein 1-7 H-atoms may be replaced by F and/or Chlorine,

X, X′ is either NH or is missing

Hal F, Cl, Br or I

Compound According to Formula II Selected from the Following Group of Compounds:

1-[4-(4-Amino-5-oxo-5H-pyrido[2,3-d]pyrimidin-8-yl)-phenyl]-3-(2-fluor-5-trifluormethyl-phenyl)-urea,

1-[4-(4-Amino-5-oxo-5H-pyrido[2,3-d]pyrimidin-8-yl)-phenyl]-3-(4-chlor-5-trifluormethyl-phenyl)-urea,

1-[4-(4-Amino-5-oxo-5H-pyrido[2,3-d]pyrimidin-8-yl)-phenyl]-3-(2,4-difluor-phenyl)-urea,

1-[4-(4-Amino-5-oxo-5H-pyrido[2,3-d]pyrimidin-8-yl)-phenyl]-3-(2,6-difluor-phenyl)-urea,

1-[4-(4-Amino-5-oxo-5H-pyrido[2,3-d]pyrimidin-8-yl)-phenyl]-3-(3-fluor-5-trifluormethyl-phenyl)-urea,

1-[4-(4-Amino-5-oxo-5H-pyrido[2,3-d]pyrimidin-8-yl)-phenyl]-3-(4-fluor-5-trifluormethyl-phenyl)-urea,

1-[4-(4-Amino-5-oxo-5H-pyrido[2,3-d]pyrimidin-8-yl)-phenyl]-3-(4-methyl-5-trifluormethyl-phenyl)-urea,

1-[4-(4-Amino-5-oxo-5H-pyrido[2,3-d]pyrimidin-8-yl)-phenyl]-3-(2,3,4,5,6-pentafluor-phenyl)-urea,

1-[4-(4-Amino-5-oxo-5H-pyrido[2,3-d]pyrimidin-8-yl)-phenyl]-3-(2,4-dibrom-6-fluor-phenyl)-urea,

1-[4-(4-Amino-5-oxo-5H-pyrido[2,3-d]pyrimidin-8-yl)-phenyl]-3-(2-fluor-6-trifluormethyl-phenyl)-urea,

1-[4-(4-Amino-5-oxo-5H-pyrido[2,3-d]pyrimidin-8-yl)-phenyl]-3-(2-fluor-5-methyl-phenyl)-urea,

1-[4-(4-Amino-5-oxo-5H-pyrido[2,3-d]pyrimidin-8-yl)-phenyl]-3-(2,3,4-trifluor-phenyl)-urea,

1-[4-(4-Amino-5-oxo-5H-pyrido[2,3-d]pyrimidin-8-yl)-phenyl]-3-(4-brom-2,6-difluor-phenyl)-urea,

1-[4-(4-Amino-5-oxo-5H-pyrido[2,3-d]pyrimidin-8-yl)-phenyl]-3-(2-fluor-3-trifluormethyl-phenyl)-urea,

1-[4-(4-Amino-5-oxo-5H-pyrido[2,3-d]pyrimidin-8-yl)-phenyl]-3-[2-(1-tert-butyloxycarbonyl-piperidin-4-yl)-phenyl]-urea,

N-[4-(4-Amino-5-oxo-5H-pyrido[2,3-d]pyrimidin-8-yl)-phenyl]-2,4-dichlor-benzamid,

N-[4-(4-Amino-5-oxo-5H-pyrido[2,3-d]pyrimidin-8-yl)-phenyl]-4-chlor-5-trifluormethyl-benzamid,

N-[4-(4-Amino-5-oxo-5H-pyrido[2,3-d]pyrimidin-8-yl)-phenyl]-2-fluor-5-trifluormethyl-benzamid,

1-[4-(4-Amino-5-oxo-5H-pyrido[2,3-d]pyrimidin-8-yl)-phenyl]-3-[3-chlor-5trifluormethyl-2-(piperidin-4-yloxy)-phenyl]-urea,

1-[4-(4-Amino-5-oxo-5H-pyrido[2,3-d]pyrimidin-8-yl)-phenyl]-3-[(2-fluor-5-(2-dimethylamino-ethoxy)-phenyl]-urea,

1-[4-(4-Amino-5-oxo-5H-pyrido[2,3-d]pyrimidin-8-yl)-phenyl]-3-[5-fluor-2(piperidin-4-yloxy)-phenyl]-urea,

1-[4-(4-Amino-5-oxo-5H-pyrido[2,3-d]pyrimidin-8-yl)-phenyl]-3-[4-chlor-5-trifluormethyl-2-(piperidin-4-yloxy)-phenyl]-urea,

1-[4-(4-Amino-5-oxo-5H-pyrido[2,3-d]pyrimidin-8-yl)-phenyl]-3-[2-(piperidin-4-yloxy)-phenyl]-urea,

1-[4-(4-Amino-5-oxo-5H-pyrido[2,3-d]pyrimidin-8-yl)-phenyl]-3-[2-fluor-5-(2-diethylamino-ethoxy)-phenyl]-urea,

1-[4-(4-Amino-5-oxo-5H-pyrido[2,3-d]pyrimidin-8-yl)-phenyl]-3-[2-fluor-5-[2-(piperidin-1-yl)-ethoxy]-phenyl]-urea,

1-[4-(4-Amino-5-oxo-5H-pyrido[2,3-d]pyrimidin-8-yl)-phenyl]-3-[4-fluor-2-(2-dimethylamino-ethoxy)-phenyl]-urea,

1-[4-(4-Amino-5-oxo-5H-pyrido[2,3-d]pyrimidin-8-yl)-phenyl]-3-[4-fluor-2-(2-diethylamino-ethoxy)-phenyl]-urea,

1-[4-(4-Amino-5-oxo-5H-pyrido[2,3-d]pyrimidin-8-yl)-phenyl]-3-[3-chlor-4-[2-(morpholin-4-yl)-ethoxy]-phenyl]-urea,

1-[4-(4-Amino-5-oxo-5H-pyrido[2,3-d]pyrimidin-8-yl)-phenyl]-3-[4-fluor-2-[2-(morpholin-4-yl)-ethoxy]-phenyl]-urea,

1-[4-(4-Amino-5-oxo-5H-pyrido[2,3-d]pyrimidin-8-yl)-phenyl]-3-[3-chlor-4-(2-dimethylamino-ethoxy)-phenyl]-urea,

1-[4-(4-Amino-5-oxo-5H-pyrido[2,3-d]pyrimidin-8-yl)-phenyl]-3-[3-chlor-4-(2-diethylamino-ethoxy)-phenyl]-urea,

1-[4-(4-Amino-5-oxo-5H-pyrido[2,3-d]pyrimidin-8-yl)-phenyl]-3-[4-chlor-2-(2-dimethylamino-ethoxy)-phenyl]-urea,

1-[4-(4-Amino-5-oxo-5H-pyrido[2,3-d]pyrimidin-8-yl)-phenyl]-3-[2-chlor-5-(2-diethylamino-ethoxy)-phenyl]-urea,

### EXAMPLE 5

### SGK1 Nucleotide Polymorphism

The nucleotide sequence defining intron 6 of facultative hypertensive patients is . . . aattacattCgcaacccag . . . , whereas the nucleotide sequence representing a healthy population is . . . aattacattTgcaacccag . . . . The sequences are available through accession number GI 2463200 Position 2071.

The exon 8 sequences of facultative hypertensive patients are either homozygotic . . . tactgaCttcggact . . . or . . . tactgaTttcggact . . . or heterozygotic . . . tactgaCttcggact . . . and . . . tactgaTttcggact . . . . The sequences are available through accession number NM _{—}005627.2, Position 777.

A homozygotic individual with a TT nucleotide combination is protected even if simultaneously a CC single nucleotide polymorphism is presented in intron 6.

### EXAMPLE 6

### Expression of GluR6 in Hippocampus of dexamethasone Treated Ice and Control Mice

Age and sex matched siblings of Sv129J mice were used for this study. 2-3 month old mice were anesthetized with Ketamin (100 mg/kg BW, Sigma) and Xylazine (4 mg/kg BW, Sigma) prior to the subcutaneous implantation of a placebo or dexamethasone pellet (both from Innovative Research of America, Sarasota, USA). Dexamethasone pellets had a continuous and linear release of 238 μg Dexamethasone per day and were used for either 8 or 20 days. To obtain the brain, mice, anesthetized with the above mentioned mixture, were terminally bled into the thoracic cavity, placed on ice where the brain was then taken out of the skull and immediately frozen in liquid nitrogen.

### REFERENCES

Berk M, Plein H, Ferreira D. Platelet glutamate receptor supersensitivity in major depressive disorder. Clin Neuropharmacol. 2001;24:129-32.

Böhmer, C., Wagner, C. A., Beck, S., Moschen, I., Melzig, J., Werner, A., Lin, J.-T., Lang, F., Wehner, F. The Shrinkage-activated Na⁺ Conductance of Rat Hepatocytes and its Possible Correlation to rENaC. Cell Phys Biochem. 2000;10:187-194.

Brenan F E, Fuller P J. Rapid upregulation of serum and glucocorticoid-regulated kinase (sgk) gene expression by corticosteroids in vivo. Mol Cell Endocrinol. 2000;30;166:129-36.

Busjahn A, Aydin A, Uhlmann R, Feng Y, Luft F C, Lang F. Serum- and glucocorticoid-regulated kinase (SGK1) gene and blood pressure. Hypertension 40(3): 256-260, 2002

Chase T N, Bibbiani F, Oh J D. Striatal glutamatergic mechanisms and extrapyramidal movement disorders. Neurotox Res. 2003;5:139-146.

Chen S Y, Bhargava A, Mastroberardino L, Meijer O C, Wang J, Buse P, Firestone G L, Verrey F, Pearce D: Epithelial sodium channel regulated by aldosterone-induced protein sgk. Proc Natl Acad Sci USA 1999;96:2514-2519.

Cowling R T, Birnboim H C. Expression of serum- and glucocorticoid-regulated kinase (sgk) mRNA is up-regulated by GM-CSF and other proinflammatory mediators in human granulocytes. J Leukoc Biol. 2000;67:240-248.

Danysz W, Parsons C G. The NMDA receptor antagonist memantine as a symptomatological and neuroprotective treatment for Alzheimer's disease: preclinical evidence. Int J Geriatr Psychiatry. 2003;18:S23-32.

Davis K M, Wu J Y. Role of glutamatergic and GABAergic systems in alcoholism. J Biomed Sci. 2001;8:7-19.

De la Rosa D A, Zhang P, Naray-Fejes-Toth A, Fejes-Toth G, Canessa C M: The serum and glucocorticoid kinase sgk increases the abundance of epithelial sodium channels in the plasma membrane of Xenopus oocytes. J Biol Chem 1999;274:37834-37839.

Farber N B, Kim S H, Dikranian K, Jiang X P, Heinkel C. Receptor mechanisms and circuitry underlying NMDA antagonist neurotoxicity. Mol Psychiatry 2002;7:32-43.

Francis P T, Poynton A, Lowe S L, Najlerahim A, Bridges P K, Bartlett J R, Procter A W, Bruton C J, Bowen D M. Brain amino acid concentrations and Ca2+-dependent release in intractable depression assessed antemortem. Brain Res. 1989;494:315-324.

Frandsen A, Schousboe A. AMPA receptor-mediated neurotoxicity: role of Ca2+ and desensitization. Neurochem Res. 2003;28:1495-1499

Ghose S, Weickert C S, Colvin S M, Coyle J T, Herman M M, Hyde T M, Kleinman J E. Glutamate carboxypeptidase II gene expression in the human frontal and temporal lobe in schizophrenia. Neuropsychopharmacology. 2004;29:117-125.

Gras G, Chretien F, Vallat-Decouvelaere A V, Le Pavec G, Porcheray F, Bossuet C, Leone C, Mialocq P, Dereuddre-Bosquet N, Clayette P, Le Grand R, Creminon C, Dormont D, Rimaniol A C, Gray F. Regulated expression of sodium-dependent glutamate transporters and synthetase: a neuroprotective role for activated microglia and macrophages in HIV infection? Brain Pathol. 2003;13:211-222.

Kawahara Y, Kwak S, Sun H, Ito K, Hashida H, Aizawa H, Jeong S Y, Kanazawa I. Human spinal motoneurons express low relative abundance of GluR2 mRNA: an implication for excitotoxicity in ALS. J Neurochem. 2003;85:680-689.

Kobayashi T, Cohen P: Activation of serum- and glucocorticoid-regulated protein kinase by agonists that activate phosphatidylinositide 3-kinase is mediated by 3-phosphoinositide-dependent protein kinase-1(PDK1) and PDK2. Biochem J 1999;339:319-328.

Kobayashi T, Deak M, Morrice N, Cohen P. Characterization of the structure and regulation of two novel isoforms of serum- and glucocorticoid-induced protein kinase. Biochem. J.1999;344:189-197.

Kolson D L. Neuropathogenesis of central nervous system HIV-1 infection. Clin Lab Med. 2002;22:703-717.

Kornhuber J, Weller M. Amantadine and the glutamate hypothesis of schizophrenia. Experiences in the treatment of neuroleptic malignant syndrome. J Neural Transm Gen Sect. 1993;92:57-65.

Lang F, Cohen P. Regulation and physiological roles of serum- and glucocorticoid-induced protein kinase isoforms. Science STKE. Nov. 13, 2001;2001(108):RE17.

Lang F, Klingel K, Wagner C A, Stegen C, Wärntges S, Friedrich B, Lanzendörfer M, Melzig J, Moschen I, Steuer S, Waldegger S, Sauter M, Paulmichl M, Gerke V, Risler T, Gamba G, Capasso G, Kandolf R, Hebert S C, Massry S G, Bröer S: Deranged transcriptional regulation of cell volume sensitive kinase hSGK in diabetic nephropathy. Proc Natl Acad Sci USA 2000;97:8157-8162.

Llansola M, Erceg S, Hernandez-Viadel M, Felipo V. Prevention of ammonia and glutamate neurotoxicity by carnitine: molecular mechanisms. Metab Brain Dis. 2002;17:389-397.

Leski M L, Hassinger L C, Valentine S L, Baer J D, Coyle J T. L-type calcium channels reduce ROS generation in cerebellar granule cells following kainate exposure. Synapse. 2002;43:30-41.

Lifton R P. Molecular genetics of human blood pressure variation. Science 1996;272:676-680.

Lipsky R H, Goldman D. Genomics and variation of ionotropic glutamate receptors. Ann N Y Acad Sci. 2003;1003:22-35.

McCullumsmith R E, Meador-Woodruff J H. Striatal excitatory amino acid transporter transcript expression in schizophrenia, bipolar disorder, and major depressive disorder. Neuropsychopharmacology. 2002;26:368-375.

McDonald J W, Shapiro S M, Silverstein F S, Johnston M V. Role of glutamate receptor-mediated excitotoxicity in bilirubin-induced brain injury in the Gunn rat model. Exp Neurol. 1998;150:21-29.

Meador-Woodruff J H, Clinton S M, Beneyto M, McCullumsmith R E. Molecular abnormalities of the glutamate synapse in the thalamus in schizophrenia. Ann N Y Acad Sci. 2003;1003:75-93.

Meador-Woodruff J H, Hogg A J Jr, Smith R E. Striatal ionotropic glutamate receptor expression in schizophrenia, bipolar disorder, and major depressive disorder. Brain Res Bull. 2001;55:631-640.

Michael-Titus A T, Bains S, Jeetle J, Whelpton R. Imipramine and pheneizine decrease glutamate overflow in the prefrontal cortex—a possible mechanism of neuroprotection in major depression? Neuroscience. 2000;100:681-684.

Náray-Fejes-Tóth A, Canessa C, Cleaveland E S, Aldrich G, Fejes-Tóth G: sgk is an aldosterone-induced kinase in the renal collecting duct. Effects on epithelial Na⁺ channels. J Biol Chem 1999;274:16973-16978.

Niederberger E, Schmidtko A, Rothstein J D, Geisslinger G, Tegeder I. Modulation of spinal nociceptive processing through the glutamate transporter GLT-1. Neuroscience. 2003;116:81-87.

Park J, Leong M L, Buse P, Maiyar A C, Firestone G L, Hemmings B A: Serum and glucocorticoid-inducible kinase (SGK) is a target of the Pl3-kinase-stimulated signaling pathway. EMBO J 1999;18:3024-3033.

Riedel G, Sandager-Nielsen K, Macphail E M. Impairment of contextual fear conditioning in rats by Group I mGluRs: reversal by the nootropic nefiracetam. Pharmacol Biochem Behav. 2002;73:391-399.

Schiffer H H. Glutamate receptor genes: susceptibility factors in schizophrenia and depressive disorders? Mol Neurobiol. 2002;25:191-212.

Shigaev A, Asher C, Latter H, Garty H, Reuveny E: Regulation of sgk by aldosterone and its effects on the epithelial Na(+) channel. Am J Physiol 2000;278:F613-F619.

Staessen J A, Wang J, Bianchi G, Birkenhager W H. Essential hypertension. Lancet. 2003;361:1629-1641.

Taylor D L, Diemel L T, Cuzner M L, Pocock J M. Activation of group II metabotropic glutamate receptors underlies microglial reactivity and neurotoxicity following stimulation with chromogranin A, a peptide up-regulated in Alzheimer's disease. J Neurochem. 2002;82:1179-1191.

Taylor D L, Diemel L T, Pocock J M. Activation of microglial group III metabotropic glutamate receptors protects neurons against microglial neurotoxicity. J Neurosci. 2003;23:2150-2160.

Wagner C A, Ott M, Klingel K, Beck S, Melzig J, Friedrich B, Wild N K, Bröer S, Moschen I, Albers A, Waldegger S, Tümler B, Egan E, Geibel J P, Kandolf R, Lang F. Effects of serine/threonine kinase SGK1 on the epithelial Na+ channel (EnaC) and CFTR. Cell Physiol Biol 2001;11:209-218.

Warnock D G. Liddle syndrome: genetics and mechanisms of Na+ channel defects. Am J Med Sci. 2001;322:302-307.

Webster MK, Goya L, Ge Y, Maiyar A C, Firestone G L: Characterization of sgk, a novel member of the serine/threonine protein kinase gene family which is transcriptionally induced by glucocorticoids and serum. Mol Cell Biol 1993;13:2031-2040.

Zieminska E, Stafiej A, Lazarewicz J W. Role of group I metabotropic glutamate receptors and NMDA receptors in homocysteine-evoked acute neurodegeneration of cultured cerebella granule neurons. Neurochem Int. 2003;43:481-492.

1. A method for altering glutamate receptor activity comprising, contacting cells expressing SGK1, SGK2 or SGK3 with a substance that modulates serum and glucocorticoid inducible kinases.

2. Use of the method according to claim 1 for the preparation of a medicament for the treatment of a disease related to glutamate receptor up- or down-regulation.

3. The method according to claim 2, wherein the disease is selected from the group of: Epilepsy, stroke, posttraumatic behavioral disorders, anxiety, schizophrenia, bipolar disorders, depression, hepatic enzephalopathy, morbus hemolyticus neonatorum, addiction, alcoholisms, HIV-enzephalopathy, neurodegenerative disorders, extra pyramidal motor disturbance, ataxia, amyotroph lateral sclerosis, M. Alzheimer, macula degeneration, deafness.

4. A method for determining the progression, regression or onset of a neuropsychiatric disease by measuring the up-regulated expression of SGK1, SGK2 or SGK3 in tissue samples and specimens.

5. A method according to claim 4, wherein the SGK1 comprises a selected single nucleotide polymorph variant.

6. A method according to claim 1 for the diagnosis of disease, wherein the disease is selected from the group of: Epilepsy, anxiety, schizophrenia, bipolar disorders, mental depression, addiction, alcoholisms, neurodegenerative, extra pyramidal motor disturbance, neurodegenerative disorders, Ataxia, M. Alzheimer, Macula degeneration, deafness.

7. Use of SGK1 inhibitors selected from the listed compounds having the general formula I or II for the manufacture of a medicament for the treatment of disorders caused by dysregulated glutamate receptors.
